(19) United States
(12) Patent Application Publication
(10) Pub. No.: US 2015/0284759 A1
(43) Pub. Date: Oct. 8, 2015

(54) METHOD FOR PRODUCING D-ALLOSE

(71) Applicants: MATSUTANI CHEMICAL INDUSTRY CO., LTD., Itami-shi, Hyogo (JP); NAGASE & CO., LTD., Osaka-shi, Osaka (JP); NAGASE CHEMTEX CORPORATION, Osaka-shi, Osaka (JP)

(72) Inventors: Pushpa Kiran Gullapalli, Itami-shi (JP); Tomoya Shintani, Itami-shi (JP); Yukifumi Nishimoto, Tokyo (JP); Koichi Harazono, Fukuchiyama-shi (JP)

(73) Assignees: MATSUTANI CHEMICAL INDUSTRY CO., LTD., Itami-shi, Hyogo (JP); NAGASE & CO., LTD., Osaka-shi, Osaka (JP); NAGASE CHEMTEX CORPORATION, Osaka-shi, Osaka (JP)

(21) Appl. No.: 14/439,970

(22) PCT Filed: Oct. 30, 2013
(86) PCT No.: PCT/JP2013/079450

§ 371 Date: Apr. 30, 2015

(30) Foreign Application Priority Data
Oct. 30, 2012 (JP) 2012-239181

(51) Int. Cl. C12P 19/24; C12N 9/90; C12P 19/02; C12N 11/00

(52) U.S. Cl. CPC C12P 19/24; C12N 11/00; C12N 9/90; C12P 19/02

(57) ABSTRACT

Provided herein is an enzyme protein produced by a highly safe microorganism and having the activity to isomerize D-psicose to D-allose. The protein is any of the following proteins (a) to (c) with the activity to isomerize D-psicose to D-allose. (a) A protein comprising the amino acid sequence represented by SEQ ID NO: 1 originating in strain 710 of a microorganism of genus Streptomyces (Deposition Number: NITE BP-01423), or SEQ ID NO: 2 originating in strain 720 of a microorganism of genus Streptomyces (Deposition Number: NITE BP-01424), and having the activity to isomerize D-psicose to D-allose. (b) A protein comprising the amino acid sequence represented by SEQ ID NO: 1 or SEQ ID NO: 2 with the substitution, addition, insertion, or deletion of one or several amino acid residues, and having the activity to isomerize D-psicose to D-allose. (c) A protein comprising an amino acid sequence that is at least 60% homologous to the amino acid sequence represented by SEQ ID NO: 1 or SEQ ID NO: 2, and having the activity to isomerize D-psicose to D-allose.

### TECHNICAL FIELD

The present invention relates to novel use of proteins originating in microorganisms of genus Streptomyces and comprising the amino acid sequence represented by SEQ ID NO:1 or SEQ ID NO: 2, specifically to use of enzymes that isomerize D-psicose to D-allose.

The present invention elucidates a sugar isomerization reaction catalyzing function that was found in the enzymes produced by microorganisms of genus Streptomyces and also relates to a technique for producing D-allose by utilizing the novel function.

### BACKGROUND ART

Simple sugars are broadly classified into aldoses (sugars with an aldehyde group representing the carbonyl group) ketoses (sugars with a ketone group representing the carbonyl group), and sugar alcohols (also known as polyols; sugars without a carbonyl group) according to the state of the reducing group (carbonyl group). Simple sugars include what is known as “rare sugar.” International Society of Rare Sugars defines rare sugar as a sugar that occurs rarely in nature, and many of rare sugars are produced in only small yields even by organic chemical syntheses. For this reason, there have, been ongoing developments of processes for efficiently producing aldohexose (aldose) rare sugars, including allose, and these rare sugars are being investigated for their unique properties.

Rare sugars are known to have physiological activities. For example, an anti-tumor agent containing a derivative of D-allose as an active ingredient is disclosed (Patent Literature 1). Also disclosed as a technique that takes advantage of sugar properties against reactive oxygen species is, for example, reactive oxygen production inhibitor containing polysaccharides that have the property to inhibit reactive oxygen species (Patent Literature 2).

Psicose is a hexose with a reducing ketone group. This rare sugar has become relatively easily available after the advent of epimerase. It has been indicated that D-psicose is potentially effective as a raw material or an intermediate of products such as sweeteners, fermentation carbon sources, reagents, cosmetics, and drugs. For example, a hydantoin derivative synthesis using D-psicose as a raw material has been reported as an application of psicose used as an intermediate raw material of products such as reagents and drugs (Non Patent Literature 1).

D-allose can be produced from D-psicose. For example, patent literatures directed to producing D-allose with an enzyme isomerase capable of producing D-allose from D-psicose have been proposed, including, for example, a method for mass production of a pure rare sugar through continuous chromatographic separation of a simulated moving bed as a target rare sugar fraction from a liquid concentrate obtained by the conversion of a substrate rare sugar into a target rare sugar under the catalytic effect of an isomerization catalyzing enzyme (Patent Literature 3), and a D-allose producing method that produces D-allose through isomerization under the effect of a Pseudomonas stutzeri (IPOD FERM BP-08593)-derived protein having an L-rhamnose isomerase activity (Patent Literature 4).

There is also proposed an aldohexose producing method that produces D-allose and D-altrose from D-psicose, and L-altrose from L-psicose in D-psicose- and/or L-psicose-containing solution acted upon by D-xylose isomerase, and collects one or more aldohexoses selected from these D-allose, D-altrose and L-altrose (Patent Literature 5).

### CITATION LIST

### Patent Literature

Patent Literature 1: JP-B-59-40400
Patent Literature 2: JP-A-07-285871
Patent Literature 3: JP-A-2006-153591
Patent Literature 4: JP-A-2008-109933
Patent Literature 5: JP-A-9002-17392
Patent Literature 6: WO2007-058.086
Patent Literature 7: U.S. Pat. No. 5,620,960 (1999)
Patent Literature 8: JP-A-2009-153516

### Non Patent Literature

Non Patent Literature 1: Tetrahedron, 47, No. 12/13, p 2113 (1991)
Non Patent Literature 2: J. Bacteriol., 73:410-414 (1956).
Non Patent Literature 3: J. Biosci Bioeng., 96:89-91 (2003).
Non Patent Literature 4: Davis et al., BASIC METHODS IN MOLECULAR BIOLOGY, 1986
Non Patent Literature 5: Methods Carbohydr. Chem., 1:102-104 (1962).
Non Patent Literature 6: Carbohydr. Res., 24:192-197 (1972).
Non Patent Literature 7: J. Ferment Bioeng., 8: 539-541 (1998).
Non Patent Literature 8: J. Mol. Biol., 300:917-933 (2000).

### SUMMARY OF INVENTION

### Technical Problem

Various techniques have been developed to produce D-allose from a variety of microorganisms. However, actual applications for the production of D-allose for food have been difficult because of the safety issue posed by the microorganisms. This has created a demand for the production of rare sugar with an enzyme originating in microorganisms that do not pose a safety problem.

For rare sugar production, for example, it is known that D-psicose can be produced from D-fructose with a D-ketohexose 3-epimerase originating in Pseudomonas cichorii. However, Pseudomonas cichorii, being a plant pathogen, are not necessarily suitable for food applications. Production of D-psicose with microorganisms of genus Rhizobium has been proposed (Patent Literature 6). However, some Rhizobium microorganisms are plant pathogens, and are not desirable for food applications. Further, these techniques are directed to producing D-psicose, not D-allose.

The present invention is intended to overcome the safety issue of the related art, and it is an object of the present invention to obtain an isomerase that can catalyze the isomerization of D-psicose to D-allose in a high yield from putatively essentially non-toxic bacterial strains selected from the strains listed in the List of Existing Additives Directory Items, which contains a list of bacterial strains approved for food production. The present invention is also intended to provide a D-allose producing method that uses such an enzyme.

Yet another object of the present invention is to provide a method for producing D-allose with the use of a novel enzyme obtained from actinomycetes, which is unlikely to cause problems in food production as existing additives.

### Solution to Problem

The present inventors completed the present invention after long studies conducted to find a D-allose producing microorganism that does not involve the safety issue of the related art, specifically an enzyme produced by such a microorganism. Specifically, the present invention is based on the finding of an enzyme having the activity to convert D-psicose to D-allose and obtained from a created library of actinomycetes isolated from soil. Actinomycetes are unlikely to cause problems in food production when used as the source microorganism of the enzyme for existing additives. An isomerase obtained from actinomycetes can thus be used as a very useful means to mass produce D-allose.

Specifically, the gist of the present invention lies in the following proteins: (1) to (3) with activity.

(1) A protein of any of the following (a) to (c) with the activity to recognize and react with the C1 CHO group and the C2 OH group of an aldose, and convert the C1 CHO group to an OH group and the C2 OH group to a CO group, or the activity to recognize and react with the C1 OH group and the C2 CO group of a ketose, and convert the C1 OH group to a CHO group and the C2 CO group to an OH group,

(a) a protein comprising the amino acid sequence represented by SEQ ID NO: 1 or SEQ ID NO: 2,

(b) a protein comprising the amino acid sequence represented by SEQ ID NO: 1 or SEQ ID NO: 2 with the substitution, addition, insertion, or deletion of one or several amino acid residues,
(c) a protein comprising an amino acid sequence that is at least 60% homologous to the amino acid sequence represented by SEQ ID NO: 1 or SEQ ID NO: 2.

(2) The protein according to (1), wherein the protein isomerizes a ketose D-psicose to an aldose D-allose.

(3) The protein according to (1) or (2), wherein the protein is specified by the following physical and chemical properties (d) to (f),

(d) an effective pH of 6.0 to 11.0, and an optimum pH of 9.0,

(e) an effective temperature of 10 to 80° C., and an optimum temperature of 60° C., and

(f) reactivity to L-rhamnose D-xylose, D-ribose, D-allose, D-glucose, and L-arabinose.

The gist of the present invention also lies in the DNA of (4), the recombinant vector of (5), the host cell of (6), and the recombinant protein producing method of (7) below.

(4) A DNA of Streptomyces sp. 710 (Deposition Number: NITE BP-01423) or Streptomyces sp. 720 (Deposition Number: NITE BP-01424) origin comprising the base sequence of SEQ ID NO: 3 or SEQ ID NO: 4, a complementary sequence thereof, or a sequence with a part of or all of the base sequence or the complementary sequence, and encoding the protein of (1) or (2).

(5) A recombinant vector comprising the DNA of (4).

(6) A host cell comprising an expression system capable of causing expression of the protein of (1), (2), or (3).

(7) A method for producing a recombinant protein,

the method comprising culturing the host cell with the expression system of (6) in a medium, and collecting the recombinant protein of (1), (2), or (3) from the obtained culture.

The gist of the present invention also lies in the D-allose producing methods of the following (8) to (12).

(8) A method for producing D-allose, the method comprising isomerizing D-psicose to D-allose under the activity of the protein of (1), (2), or (3).

(9) The method for producing D-allose according to (8), wherein the D-psicose is produced by epimerizing D-fructose.

(10) The method for producing D-allose according to (8), wherein the D-psicose is produced by directing D-glucose to D-fructose through isomerization, and epimerizing the D-fructose.

(11) The method for producing D-allose according to (8), wherein the D-psicose is produced by obtaining D-glucose from an unused resource, directing the D-glucose into D-fructose through isomerization, and epimerizing the D-fructose.

(12) The method for producing D-allose according to any one of (8) to (11), wherein the target product D-allose is a mixture of D-psicose and D-allose.

### Advantageous Effects of Invention

The present invention, specifically the use of an enzyme protein originating in microorganisms of genus Streptomyces makes it possible to recognize and react with the C1 CHO group and the C2 OH group of an aldose, and convert the C1 CHO group to an OH group and the C2 OH group to a CO group, or recognize and react with the C1OH group and the C2 CO group of a ketose, and convert the C1OH group to a CHO group and the C2 CO group to an OH group, preferably produce D-allose from D-psicose. The present invention can provide an enzyme having the amino acid sequence of SEQ ID NO: 1 or SEQ ID NO: 2 originating in microorganisms of genus Streptomyces. The enzyme recognizes and reacts with the C1 CHO group and the C2 OH group of an aldose, and converts the C1 CHO group to an OH group and the C2 OH group to a CO group, or recognizes and reacts with the C1OH group and the C2 CO group of a ketose, and converts the C1OH group to a CHO group and the C2 CO group to an OH group, and preferably isomerizes D-psicose to D-allose. The present invention with the use of the enzyme enables D-allose production.

The present invention enabling use of highly safe bacteria in terms of bacterial culture is a large technological advancement. The present invention providing the enzyme capable of producing D-allose through isomerization of D-psicose, and the established method of production of such enzymes is thus considered to be industrially highly useful not only in sugar production but in related food, cosmetic, and drug industries. The present invention also enables D-allose production via D-psicose from starting materials such as D-glucose, one of the cheapest and the most widely available sugars, and fructose.

### BRIEF DESCRIPTION OF DRAWINGS

FIG. 1 is a diagram representing the recombinant isomerase configuration (restriction enzyme map) of Example 1.

FIG. 2 is a diagram representing the effect of metal ions on enzyme activity.

FIG. 3 is a diagram representing (a) the effect of temperature for optimum enzyme activity, and (b) stability after 1 hour under a maintained temperature.

FIG. 4 is a diagram representing (a) the effect of pH for optimum enzyme activity, and (b) enzyme stability after 24 hours under a maintained temperature.

FIG. 5 is a diagram representing the substrate specificity of the enzymes of the present invention.

FIG. 6 is a diagram representing the result of HPLC analysis before (D-psicose) and after (a mixture of D-psicose, D-altrose, and D-allose) the reaction.

### DESCRIPTION OF EMBODIMENTS

D-allose represents a very useful rare sugar, and has an important role in inhibition of cancer cell proliferation. Arnold and Silady have reported that D-allose essentially inhibits production of segmented neutrophils, and lowers platelet numbers without causing other harmful clinical effects (Patent Literature 7). There is also a report of D-allose inhibiting reactive oxygen production (Non Patent Literature 3). D-allose also has been investigated in many studies, particularly with regard to its anticancer effect in the recent years. Under these circumstances, the present inventors conducted intensive studies to provide a technique that can be used to safely produce the useful D-allose in large quantities, with the goal of obtaining an enzyme that converts D-psicose into D-allose from bacterial strains that are safe to use in food production.

L-Rhamnose isomerase (L-RhI, E.C: 5.3.1.14) is an enzyme that catalyzes the isomerization reaction of D-psicose into D-allose. This enzyme, in Escherichia coli, is known to catalyze the reaction that reversibly isomerizes L-rhamnose into a corresponding ketose L-rhamnulose (Non Patent Literature 2). However, Escherichia coli are not necessarily safe for food production.

In order to establish a technique that enables easy production of the industrially promising D-allose, the present inventors created a library of isolated actinomycetes from soil, and searched the library for bacteria that produce an isomerase that converts D-psicose into D-allose.

The protein used as the enzyme that produces D-allose from D-psicose in the present invention is a Streptomyces microorganism-derived protein having the amino acid sequence of SEQ ID NO: 1 or SEQ ID NO: 2 with the activity to convert D-psicose into D-allose.

Typically, the enzyme of interest may be extracted from cultured bacteria, and directly used for the reaction. It is, however, preferable to immobilize the enzyme before use. The enzyme having the foregoing activity and to be immobilized may itself not be necessarily required to be purified to a high purity level as may be decided according to the intended use, and may be used as a crude enzyme. Specifically, the crude enzyme may be, for example, the microorganism itself that is capable of producing the enzyme having the activity, or a culture or a partially purified culture of such microorganisms.

The enzyme of interest for the present invention may be used after being immobilized by using an ordinary method, for example, such as a carrier binding method, a crosslinking method, and an entrapment method. These are not limited to the enzyme, and may be obtained by immobilizing the bacteria itself or the crude enzyme on a carrier such as resin. The immobilization can produce an immobilized enzyme that can remain stable for several months, as opposed to about one week without immobilization.

For example, a D-psicose solution containing 50% ethanol may be passed through an immobilized enzyme and/or an immobilized microorganism obtained after immobilizing the enzyme and/or microorganism of interest for the present invention by using a covalent bonding method as an example of the carrier binding method, and the temperature may be controlled, for example, at 42° C. for the reaction, and 4° C. for the crystallization, to continuously produce D-allose crystals. The filtrate after the crystallization may be passed again through the immobilized enzyme and/or immobilized microorganism without having the ethanol removed or being concentrated to continuously produce D-allose.

This is innovative in the sense that the method allows separation of only the D-allose by simple addition of ethanol to the mixed solution of D-psicose and D-allose. The method also does not require removing the buffer used for the enzyme reaction, and is highly advantageous in that the separation process is much less laborious, and can be performed with improved efficiency. An enzyme reaction that uses 50% D-psicose as the starting material of D-allose production yields the product as a mixed solution of 35% D-psicose and 15% D-allose, and the D-psicose and D-allose in the enzyme reaction product can be quickly separated to obtain D-allose in high purity.

As an example, the enzyme reaction performed in the present invention for the production of D-allose from D-psicose is typically performed under the following conditions. Typically, an aqueous solution of D-psicose is used as a substrate solution, and the substrate concentration is appropriately selected from 1 to 60 w/v %, desirably 10 to 50 w/v %, further desirably 20 to 40 w/v %. The enzyme reaction temperature is selected from a temperature range that does not deactivate the enzyme, for example, 10 to 85° C., desirably 40 to 80° C., further desirably the optimum temperature of 60° C. For the same reason, the enzyme reaction pH is selected from 6.0 to 11.0, more desirably the optimum pH of 9.0. The enzyme activity is selected from a range of 1 unit or more, desirably 50 to 5,000 units per gram of substrate. The reaction time depends on conditions such as the substrate amount, the enzyme amount, the temperature, and the pH. Taking as an example a reaction performed in a batch instead of using the immobilized enzyme, it is desirable for economy that the reaction be performed for about 2 to 100 hours, more desirably about 5 to 50 hours.

The present invention is concerned with a D-allose producing method that produces D-allose through isomerization of D-psicose acted upon by any of the following proteins (a) to (c) having activity. The method produces D-allose from D-psicose by using an enzyme produced by highly safe strains of actinomycetes and capable of producing D-allose from D-psicose.

(a) A protein with the activity comprising the amino acid sequence of SEQ ID NO: 1 or SEQ ID NO: 2.

(b) A protein with the activity comprising the amino acid sequence of SEQ ID NO: 1 or SEQ ID NO: 2 with the substitution, addition, insertion, or deletion of one or several amino acid residues.

(c) A protein with the activity comprising an amino acid sequence that is at least 60% homologous to the amino acid sequence of SEQ ID NO: 1 or SEQ ID NO: 2.

The L-rhamnose isomerase activity of the proteins is specified by the following physical and chemical properties (d) to (f).

The enzyme activity of the present invention is specified by the following physical and chemical properties (d) to (f).

(d) Effective pH and optimum pH

The effective pH is 6.0 to 11.0, and the optimum pH is 9.0.

(e) Effective temperature and optimum temperature

The effective temperature is 10 to 80° C., and the optimum temperature is 60° C.

(f) Reactivity to L-rhamnose, D-xylose, D-ribose, D-allose, D-glucose, and L-arabinose.

### [D-Psicose]

The D-psicose used in the present invention may be produced by using various methods, including, for example, a method that epimerizes D-fructose, a method that directs D-glucose to D-fructose through isomerization, and epimerizes the D-fructose, and a method that obtains D-glucose from an unused resource, directs the D-glucose to D-fructose through isomerization, and epimerizes the D-fructose.

The present invention is described below in greater detail using Examples. It should be noted, however, that the present invention is in no way limited by the following Examples.

### Example 1

### Obtaining Bacterial Strains that Produce Enzyme Capable of D-Psicose to D-Allose Conversion

Soil (0.1 g) collected from an environment was suspended in 1 mL of sterile distilled water. The suspension was diluted 50 times with sterile distilled water, and 0.1 mL of the diluted suspension was inoculated on an actinomycete isolation medium, and cultured at 30° C. for 3 days. After culture, the resulting colonies were picked up (about 200 strains). The actinomycete medium of the composition presented in Table 1 was used as the actinomycete isolation medium.

TABLE 1

| Actinomycete medium | (g/L) |
|---|---|
| Potato starch | 1.0 |
| K₂HPO₄ | 0.03 |
| NaCl | 0.5 |
| MgSO₄•7H₂O | 0.1 |
| NaNO₃ | 0.1 |
| Agar | 20 |

Subsequently, the about 200 bacterial strains thus obtained were each inoculated in 5 mL of the producing medium below in a test tube, and shake cultured at 28° C., 220 rpm shaking conditions. On day 7 of culture, each culture (15 mL) was collected into a centrifuge tube, and centrifuged at 9000 rpm for 10 minutes with a HITACHI High-Speed Micro Centrifuge (Model CF15RXII). After discarding the supernatant, the bacteria were washed once with distilled water, and recentrifuged. After discarding the supernatant, the bacteria were each suspended in 2 mL of a 40 mM tris-maleate buffer (pH 7.0), and sonicated twice, each for 20 seconds, with an Astrason Ultrasonic Cell Disrupter (W385; HEAT SYSTEM) operated in a 50% duty cycle at an output control level of 5. The disrupted solution was centrifuged at 9000 rpm for 10 minutes, and the supernatant was collected to obtain a crude enzyme solution. The crude enzyme solution was adjusted to a protein concentration of 0.10 mg/50 μL, and used as an enzyme sample for the enzyme activity measurement below.

### [Composition of Producing Medium]

A sugar medium (1.0% D-psicose, 0.1% D-tagatose, 0.1% glycerol) and a mineral medium (0.26% ammonium sulfate, 0.24% potassium dihydrogen phosphate, 0.56% dipotassium hydrogen phosphate, 0.01% magnesium sulfate heptahydrate, 0.05% yeast extract; pH 7.0) are each sterilized at 121° C. for 20 minutes, and aseptically mixed in equal amounts.

### [Enzyme Activity Checking Methods]

The enzyme samples obtained as above were each checked for isomerase activity by using a HPLC method and/or a cysteine carbazole method.

### HPLC Method:

An enzyme reaction was performed with the composition presented in Table 2. After quenching the enzyme reaction by processing the reaction mixture at 100° C. for 2 minutes, the sample was cooled to room temperature, and subjected to a purification process with an ion-exchange resin and a filter to prepare a sample for HPLC. The HPLC sample was fed to a CK08EC column (80° C.; Mitsubishi Chemical Corporation), and eluted with purified water at a flow rate of 0.4 mL/min. The generated sugar composition was then measured with a Tosoh Detector RI-8020.

### Cysteine Carbazole Method:

An enzyme reaction was performed with the composition presented in Table 2. After quenching the enzyme reaction by processing the reaction mixture at 100° C. for 2 minutes, the sample was cooled to room temperature, and 50 μL of 10% trichloroacetic acid was added to 500 μL of the enzyme reaction solution to quench the reaction. The sample (550 μL) was maintained at 20° C. for 1 minute after adding 100 μL of a cysteine solution and 3 mL of 70% sulfuric acid thereto, and heated at 35° C. for 20 minutes after adding 100 μL of a carbazole solution thereto. The generated sugar composition was then measured by reading the absorbance at 540 nm.

TABLE 2
Reaction Conditions for Checking Enzyme Activity
Reaction Conditions

| | | |
|---|---|---|
| Buffer (Gly-NaOH) | 350 | μL |
| Substrate (50 mM D-psicose) | 50 | μL |
| Enzyme sample (0.10 mg/50 μL) | 50 | μL |
| Reaction temperature | 60° | C. |
| Reaction time | 10 | min |

Two strains with the desired high enzyme activity (Streptomyces sp. 710 strain, and Streptomyces sp. 720 strain; the genus was identified from the 16S rRNA base sequence) were obtained after the foregoing series of procedures. These bacterial strains Streptomyces sp. 710 and Streptomyces sp. 720 have been internationally deposited at the National Institute of Technology and Evaluation Patent Microorganism Depositary (NITE; 2-5-8 Kazusa Kamatari, Kisarazu, Chiba, Japan) with the deposition numbers NITE BP-01423 and NITE BP-01424, respectively (original deposition date: Oct. 10, 2012), under the Budapest Treaty, and are available therefrom. The 710 and 720 strains originally deposited therein were transferred to the international depositary under the Budapest Treaty upon filing requests on Oct. 7, 2013, and Oct. 30, 2013, respectively, and have been received thereby. The proof of receipt of international depositary for these two strains was issued on Oct. 30, 2013.

### Example 2

Shotgun cloning was performed for the Streptomyces sp. 710 and 720 strains.

### [Preparation of Streptomyces sp. 710 (or 720) Strain Chromosomes]

Streptomyces sp. 710 (or 720) strain was cultured at 30° C. for 2 days under 160 rpm in a tryptic soy broth medium (Becton, Dickinson and Company) supplemented with 0.5% glycine (50 mL). The culture was centrifuged (3,000 rpm, 10 min, 4° C.), and the bacteria were collected. After washing the bacteria with TS buffer (10.3% sucrose, 50 mM Tris buffer (pH 8.0), 25 mM EDTA.2Na) (30 mL), the sample was recentrifuged (3,000 rpm, 10 min, 4° C.), and the bacteria were collected. The bacteria were then allowed to stand at 37° C. for 60 minutes after adding a TS buffer (10 mL) containing 0.5% lysozyme and 0.002% N-acetylmuramidase. Thereafter, a 10% SDS aqueous solution (2.4 mL), and a TS buffer (0.4 mL) containing 2 mg/mL of proteinase K were added to the protoplast-like bacteria solution, and the mixture was slowly stirred, and allowed to stand at 37° C. for 60 minutes, and at 50° C. for 30 minutes. To the mixture were then added a 5 M sodium chloride aqueous solution (2 mL), and a 0.7 M sodium chloride aqueous solution (1.6 mL) of 10% cetyltrimethylammonium bromide that had been heated to 65° C. The mixture was slowly stirred, and allowed to stand at 65° C. for 20 minutes. Thereafter, a chloroform:isoamylalcohol solution (24:1, 20 mL) was added, and the mixture was stirred with a pipette. After centrifugation (12,000 rpm, 10 min, 4° C.), isopropyl alcohol was added to the upper layer in 0.6 times the amount of the upper layer, and the resulting precipitate was collected. The precipitate was washed with 70% ethanol, and vacuum dried. After the vacuum drying, the precipitate was dissolved in 10 mL of a TE buffer (10 mM Tris buffer, pH 8.0; 1 mM EDTA.2Na) containing 0.5 mg of Rnase to prepare a chromosome solution of Streptomyces sp. 710 (or 720) strain (preserved at 4° C.).

### Example 3

### Partial Decomposition of Chromosomes

A 10×H buffer (Takara Bio; 4.6 μL), and 0.25 units of restriction enzyme Sau3AI were added to the chromosome solution of Streptomyces sp. 710 (or 720) strain (40.4 μL), and the mixture was allowed to stand for 40 minutes at 37° C. After adding a 0.5 M EDTA aqueous solution (50 μL), the sample was electrophoresed on a 0.5% low-melting-point agarose gel, and an agarose gel fraction containing DNA fragments of 23 kb or more was cut out. The cut gel block was put in distilled water (50 mL), and was slowly shaken for 15 minutes. After repeating the same procedure, a 10×β-agarase buffer was added until the final concentration was 1× with respect to the weight of the gel block, and the gel was allowed to stand at 68° C. until it melted. The molten gel was allowed to stand for 10 minutes at 40° C., and β-agarase (3 units per 0.5 g of the gel) was added. The gel was then allowed stand for 60 minutes at 40° C. After adding a 5 M sodium chloride aqueous solution to make the final concentration 0.5 M, the mixture was allowed to stand on ice for 15 minutes. After centrifugation (15,000 rpm, 15 min, 4° C.), ethanol was added to the supernatant in 3 times the amount of the supernatant. The mixture was allowed to stand at −80° C. for 10 minutes, and centrifuged (15,000 rpm, 15 min, 4° C.). Thereafter, 70% ethanol was added to the resulting precipitate, and the mixture was centrifuged (15,000 rpm, 15 min, 4° C.). The precipitate was vacuum dried, and dissolved in distilled water (8 μL). The mixture was allowed to stand for 60 minutes at 37° C. after adding a 10× alkaline phosphatase buffer (1 μL) and alkaline phosphatase (1 μL). Thereafter, distilled water (90 μL) and a phenol:chloroform:isoamylalcohol solution (25:24:1, 100 μL) were added, and the mixture was stirred, and centrifuged (15,000 rpm, 10 min, 25° C.). Ethanol was then added to the upper layer in 3 times the amount of the upper layer. The mixture was allowed to stand at −80° C. for 10 minutes, and centrifuged (15,000 rpm, 15 min, 4° C.). Thereafter, 70% ethanol was added to the resulting precipitate, and the mixture was centrifuged (15,000 rpm, 15 min, 4° C.). The precipitate containing the partially decomposed DNA fragments of the Streptomyces sp. 710 (or 720) strain chromosomes was vacuum dried, and dissolved in distilled water (5 μL).

### Example 4

### Creation of Streptomyces sp. 710 (or 720) Strain Chromosome Cosmid Library

Ten micrograms of Actinomycete-Escherichia coli shuttle cosmid vector pTOYAMAcos (Hiroyasu Onaka et al., J. Antibiotics, 2003, Vol. 56, pp. 950-956) was decomposed with restriction enzyme BamHI, and was electrophoresed on an agarose gel. An agarose gel fraction containing a 8.3 kb DNA fragment was then cut out from the gel. The DNA fragment was purified from the cut gel block with a Geneclean Kit (Q-Biogene), and dissolved in distilled water (2.5 μL). To this was added the partially digested DNA fragment aqueous solution (5 μL) of the Streptomyces sp. XP-710 and XP-720 strain chromosomes obtained in the Example, and the mixture was allowed to stand for 17 hours at 16° C. after adding a ligation kit (Takara Bio; 7.5 μL). The plasmid in the ligation solution was then introduced into conjugative Escherichia coli by using a Gigapack III Packaging Extract Kit (Stratagene). The Escherichia coli with the plasmid was cultured at 30° C. for 2 days in an LB agar medium supplemented with carbenicillin (50 μg/L), and 1,000 colonies collected from the grown transformant (Escherichia coli) were cultured at 30° C. for 1 day in a separate LB agar medium supplemented with carbenicillin (50 g/L).

### Example 5

### Introduction of Plasmid from Escherichia coli Cosmid Library into Actinomycete

The 1,000 colonies of Escherichia coli with the cosmid plasmid from Example 4 were each cultured at 30° C. in an LB medium (5 mL) supplemented with kanamycin (50 g/mL) until the absorbance at 660 nm was 0.6. The culture (2 mL) was transferred to a sterilized Eppendorf tube, and centrifuged (3,000 rpm, 5 min, 4° C.). After decanting the supernatant, an LB medium (1 mL) was added, and the culture was vigorously agitated to wash the bacteria. The culture was centrifuged (3,000 rpm, 5 min, 4° C.), and the supernatant was decanted. This procedure was repeated two more times. After adding an LB medium (0.5 mL), the bacteria were suspended therein. Thereafter, a spore suspension (1 μL) of Streptomyces lividans 1326 strain (NBRC Number: 15675), and a suspension (0.1 mL) of the conjugative Escherichia coli with the plasmid were thoroughly mixed in a separately prepared sterilized Eppendorf tube, and spread over an actinomycete medium Daigo No. 4 (Nihon Pharmaceutical Co., Ltd.). After culturing the cells at 30° C. for 18 hours, a nutrient broth (Becton, Dickinson and Company) agar medium (agar 0.5%, 3 mL) supplemented with thiostrepton (167 g/mL) and nalidixic acid Na (67 μg/mL) was layered, and the culture was continued for 3 days to obtain grown colonies (each colony obtained came from a single Escherichia coli cell).

### Example 6

### Obtaining Recombinant Actinomycete Capable of Isomerase Production

The 1,000 colonies produced in Example 5 were each inoculated in 50 mL of the foregoing medium in a 500-mL baffled flask, and rotary cultured under 28° C., 160 rpm conditions. On day 3 of culture, each culture was collected into a 50-mL centrifuge tube. The cells were centrifuged at 9000 rpm for 10 minutes with a HITACHI High-Speed Micro Centrifuge (Model CF15RXII), and the supernatant was discarded. The bacteria were washed once with distilled water, and recentrifuged.

After discarding the supernatant, the bacteria were each suspended in 30 ml of a 40 mM tris-maleate buffer (pH 7.0), and sonicated twice, each for 20 seconds, with an Astrason Ultrasonic Cell Disrupter (W385; HEAT SYSTEM) operated in a 50% duty cycle at an output control level of 5. The disrupted solution was centrifuged at 9000 rpm for 10 minutes, and the supernatant was collected. The liquid was then membrane filtered with a Minisart (1.2 μm; Sartorius) to obtain a crude enzyme solution (for 1,000 colonies).

These crude enzyme solutions were checked for activity by using the method described above. The result confirmed activity in the crude enzyme solution of one of the colonies.

An analysis of the DNA sequences of the cosmid library from this colony found a structural gene sequence of uncertain function. An experiment was thus conducted to express this structural gene.

### Example 7

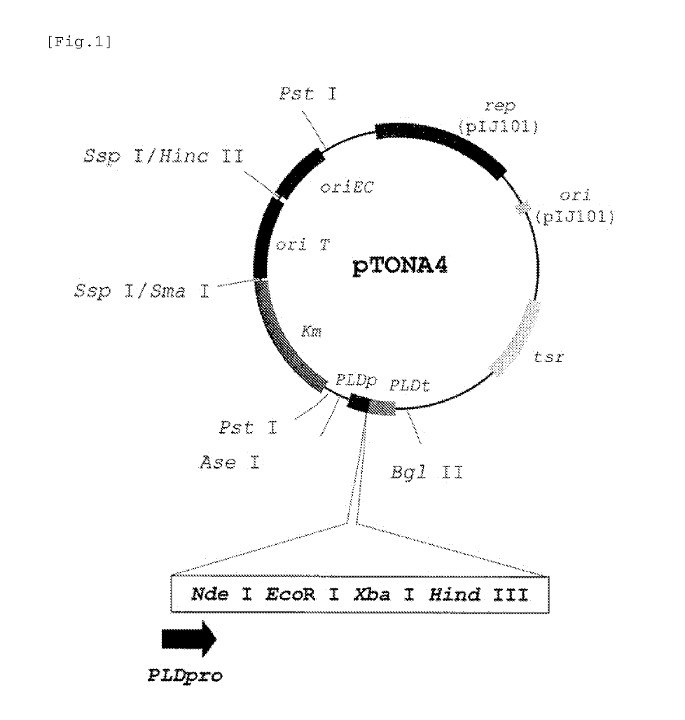

In this Example, a PCR fragment was introduced into a conjugative actinomycete plasmid pTONA4 (Patent Literature 8), and expressed in Streptomyces lividans 1326 strain (NBRC Number: 15675) to obtain the recombinant isomerase configuration (restriction enzyme map) shown in FIG. 1.

Genomic DNA was isolated from each of the Streptomyces sp. 710 and 720 stains by using an InstaGene™ Matrix (BIO-RAD). A forward primer (SEQ ID NO: 5) and a reverse primer (SEQ ID NO: 6) were synthesized. These two primers were used to perform a PCR reaction, using the genomic DNA of the Streptomyces sp. 710 strain as a template. The PCR reaction was performed with a Phusion DNA Polymerase (FINNZYMES) in 35 cycles, each cycle consisting of 98° C. for 10 seconds, 58° C. for 10 seconds, and 72° C. for 2 minutes, in sequence. The resulting PCR fragment of the PCR reaction was processed with restriction enzymes NdeI and HindIII. The restriction enzymes NdeI and HindIII were also used to process the conjugative actinomycete plasmid pTONA4. The obtained two fragments were ligated, and introduced into Streptomyces lividans 1326 strain (NBRC Number: 15675) to obtain an XGP-710 strain. It was confirmed that the resulting PCR fragment was identical to SEQ ID NO: 3 upon determining its base sequence in a DNA sequencer analysis.

Separately, a forward primer (SEQ ID NO: 7) and a reverse primer (SEQ ID NO: 8) were synthesized. These two primers were used to perform a PCR reaction, using the genomic DNA of Streptomyces sp. 720 strain as a template. An XGP-720 strain was obtained after the same procedures performed as above. It was confirmed that the resulting PCR fragment was identical to SEQ ID NO: 4 upon determining its base sequence in a DNA sequencer analysis. The primers of SEQ ID NOS: 5 to 8 are presented in Table 3.

Primers of SEQ. ID NO 5 to 8

| | | |
|---|---|---|
| SEQ ID | XGP-710 | ggaattccatatgaccgagctcgccgcggt |
| NO: 5 | primer (1) | |
| SEQ ID | XGP-710 | cccaagcttctacgccccccacccggcctg |
| NO: 6 | primer (2) | |
| SEQ ID | XGP-720 | ggaattccatatgacgagctcgccgcggt |
| NO: 7 | primer (1) | |
| SEQ ID | XGP-720 | cccaagctttcacgctccccagcccgctg |
| NO: 8 | primer (2) | |

Restriction enzymes: NdeI and HindIII

Host cells with the expression system above were cultured to obtain a crude enzyme solution.

Subsequently, the XGP-710 and XGP-720 strains were each inoculated in a TSB medium (50 mL) in a 500-mL baffled flask, and rotary cultured under 28° C., 160 rpm conditions. On day 3 of culture, each culture was collected into a 50-mL centrifuge tube. The cells were centrifuged at 9000 rpm for 10 minutes with a HITACHI High-Speed Micro Centrifuge (Model CF15RXII), and the supernatant was discarded. The bacteria were washed once with distilled water, and recentrifuged. After discarding the supernatant, the bacteria were each suspended in 30 ml of a 40 mM tris-maleate buffer (pH 7.0), and sonicated twice, each for 20 seconds, with an Astrason Ultrasonic Cell Disrupter (W385; HEAT SYSTEM) operated in a 50% duty cycle at an output control level of 5. The disrupted solution was centrifuged at 9000 rpm for 10 minutes, and the supernatant was collected. The liquid was then membrane filtered with a Minisart (1.2 μm; Sartorius) to obtain a crude enzyme solution (an XGP-710 crude enzyme solution, and an XGP-720 crude enzyme solution).

### Example 8

D-Allose can be produced by using a chemical or a biological method. Chemically, D-allose can be produced from D-ribose (Non Patent Literature 5), or through reduction of 1,2:5,6-di-O-isopropylidene-α-D-ribohexofuranose-3-ulose (Non Patent Literature 6). Enzymatically, D-allose can be produced from D-psicose with L-riboseisomerase (L-RhI) from Pseudomonas stutzerii LL172 (Non Patent Literature 7).

In the present invention, the characteristics of the isomerase obtained from Streptomyces were examined.

### (1) Enzyme Reactivity

First, the enzyme was examined for its reactivity to different substrates.

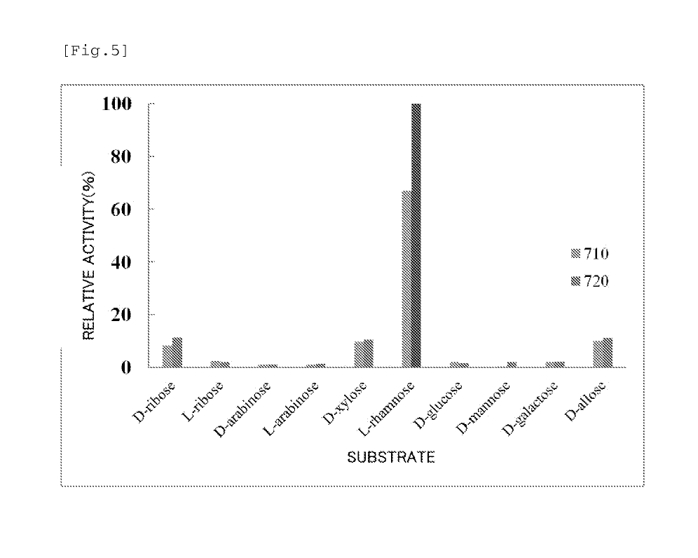

Aldoses were used as substrates. Specifically, L-rhamnose, D-ribose, D-arabinose, L-arabinose, D-xylose, D-glucose, and D-allose were reacted under the foregoing enzyme activity reaction conditions, and the resulting reaction mixtures were each measured for enzyme activity by using the cysteine carbazole method. The enzyme showed the highest substrate activity to L-rhamnose, and higher to D-xylose, D-ribose, D-allose, D-glucose, and L-arabinose (FIG. 5).

### (2) Effect of Metals on Enzyme Activity

Subsequently, in order to examine the effect of metal ions on isomerase activity, the enzyme was partially dialyzed to measure the enzyme activity. For dialysis, the crude enzyme solution was applied to a cellulose film, and immersed in a glycine-NaOH buffer containing 20 mM EDTA (pH 9.0), and the buffer was slowly stirred over a time period of 16 hours to eliminate the effect of other metal ions. The enzyme activity of the resulting apoenzyme was then measured by using the cysteine carbazole method after performing reaction in the presence of various divalent ions (under the reaction conditions presented in Table 4).

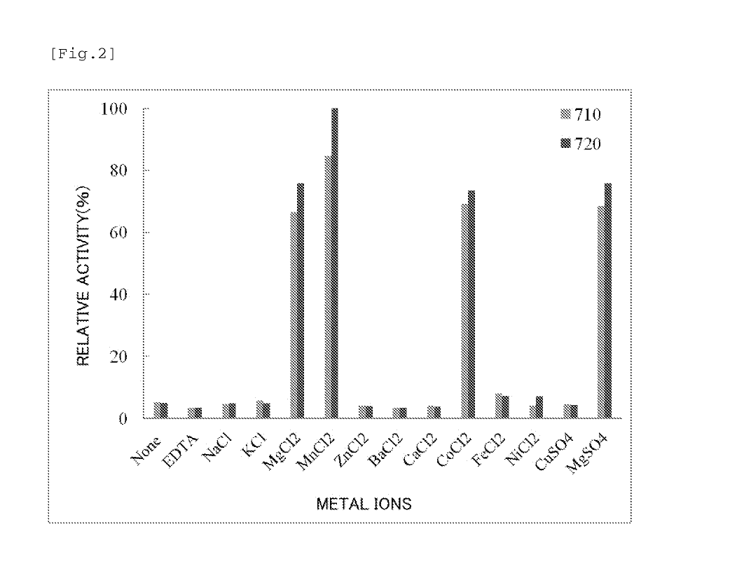

It was found that MnCl₂ greatly increased the enzyme activity. On the other hand, MgSO₄, MgCl₂, and CoCl₂ only slightly increased the activity, though the enzyme activity was shown to be metal dependent. CaCl₂, BaCl₂, ZnCl₂, and CuSO₄ inhibited the activity (FIG. 2).

It is reported that L-RhI expressed in E. coli requires Mn²⁺ or Zn²⁺ to exhibit its enzyme activity (Non Patent Literature 8).

TABLE 4
Reaction Conditions for Checking the Effect of Metal Ions
Reaction Conditions

| | | |
|---|---|---|
| Buffer (Gly-NaOH) | 350 | μL |
| Substrate (50 mM L-rhamnose) | 50 | μL |
| Dialysis Enzyme (0.10 mg/50 μL) | 50 | μL |
| Coenzyme (10 mM of each metal salt) | 50 | μL |
| Reaction temperature | 60° | C. |
| Reaction time | 10 | min |

### (3) Effect of Temperature on Enzyme Activity (Optimum Temperature)

The effect of temperature on enzyme activity was then examined.

Enzyme reactions were performed under varying reaction temperatures 10, 20, 30, 40, 50, 60, 70, 80, and 90° C. under the conditions of Table 5, and the enzyme activity was determined through measurements using the cysteine carbazole method.

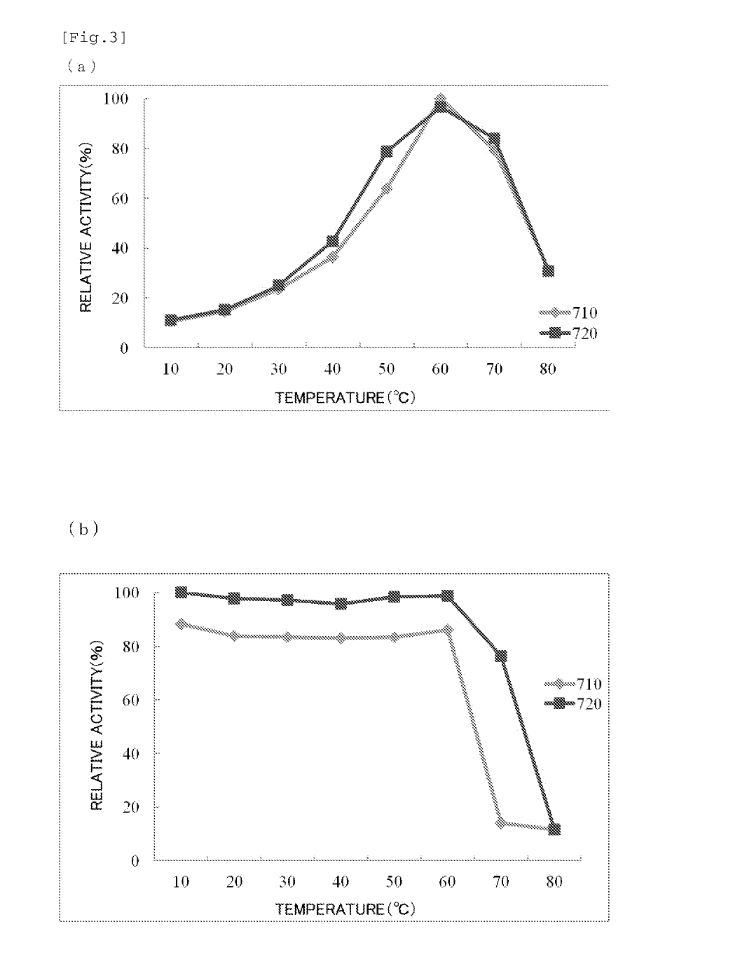

The optimum temperature for the enzyme activity was found to be 60° C. (FIG. 3a).

TABLE 5
Reaction Conditions for Checking the Effect of Temperature
Reaction Conditions

| Buffer (Gly-NaOH) | 350 | μL |
|---|---|---|
| Substrate (50 mM L-rhamnose) | 50 | μL |
| Enzyme solution (0.10 mg/50 μL) | 50 | μL |
| Coenzyme (10 mM MnCl₂) | 50 | μL |
| Reaction temperature | Varying temperatures | |
| Reaction time | 10 | min |

### (4) Heat Resistance of Enzyme Activity

The heat resistance of the enzyme activity was then examined.

Each enzyme solution was maintained in a glycine-NaOH buffer (pH 9.0) for 1 hour under different temperature conditions (10, 20, 30, 40, 50, 60, 70, 80° C.), and the remaining enzyme activity was measured by using the cysteine carbazole method after the enzyme reaction performed under the foregoing conditions.

About 80 to 90% enzyme activity remained even when maintained for 1 hour at 60° C. (FIG. 3b).

### (5) Effect of pH on Enzyme Activity (Optimum pH)

The effect of pH on enzyme activity was then examined.

Specifically, the enzyme solution was maintained in different pH buffers (50 mM citrate buffer (pH 3.0-6.0), 50 mM sodium phosphate buffer (pH 6.0-8.0), 50 mM tris-hydrochloride buffer (pH 7.0-9.0), and 50 mM glycine-sodium hydroxide buffer (pH 9.0-11.0)) at 4° C. for 24 hours. Enzyme reactions were performed under the reaction conditions of Table 6, and the remaining enzyme activity was measured by using the cysteine carbazole method. The D-allose to D-psicose isomerization reaction conditions are presented in the table below.

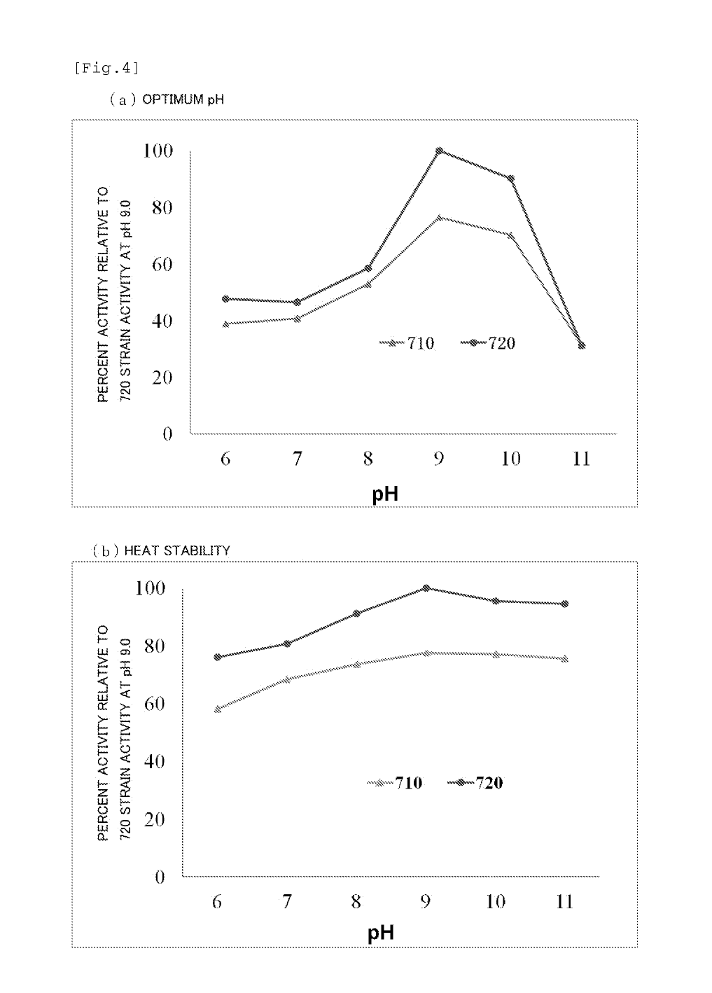

It was found that the optimum pH for the enzyme was 9.0 (FIG. 4a), and the enzyme activity was more stable in the pH 9.0 glycine-sodium hydroxide buffer (FIG. 4b).

TABLE 6
Reaction Conditions for Checking the Effect of pH
Reaction Conditions

| | | |
|---|---|---|
| Buffer (Gly-NaOH) | 350 | μL |
| Substrate (50 mM L-rhamnose) | 50 | μL |
| Enzyme solution after left for 24 hours at 4° C. | 50 | μL |
| Coenzyme (10 mM MnCl₂) | 50 | μL |
| Reaction temperature | 60° | C. |
| Reaction time | 10 | min |

### (6) Percentage Yield of Rare Sugar at the Equilibrium Point of Enzyme Catalyzed Reaction

The enzyme was then reacted with each of the aldose substrates L-rhamnose, D-ribose, D-arabinose, L-arabinose, D-xylose, D-glucose, and D-allose under the reaction conditions of Table 7 (the reaction time was 6 to 48 hours), and the sugar composition of the reaction liquid at the equilibrium point was examined by HPLC.

TABLE 7
Reaction Conditions for Different
Aldose Substrates until Equilibrium Point
Reaction Conditions

| | | |
|---|---|---|
| Buffer (Gly-NaOH) pH: 9.0 | 200 | μL |
| Substrate (0.2M of each aldose) | 375 | μL |
| Enzyme solution (0.10 mg/50 μL) | 100 | μL |
| Coenzyme (0.01M MnCl₂) | 75 | μL |
| Temperature | 55° | C. |
| Reaction time | 6 to 48 | hours |

Table 8 presents the percentage yield of ketose and aldose from each aldose acted upon by the enzyme (XGP-720 enzyme solution).

TABLE 8

| Substrate | Product^{a} | | Percentage | |
|---|---|---|---|---|
| Aldose | Ketose | Aldose | Yield^{b} (%) | Time (hr) |
| L-Rhamnose | L-Rhamnulose | None | 60:40:0 | 6 |
| D-Ribose | D-Ribulose | D-Arabinose | 65:22:13 | 24 |
| D-Arabinose | D-Ribulose | D-Ribose | 91:2:7 | 24 |
| L-Arabinose | L-Ribulose | None | 99:1:0 | 48 |
| D-Xylose | D-Xylulose | D-Lyxose | 82:16:2 | 24 |
| D-Glucose | D-Fructose | None | 83:17:0 | 48 |
| D-allose | D-Psicose | D-Altrose | 33:66:1 | 24 |

^{a}Ketose was produced first in all reactions
^{b}Percentage yield represents aldose (remaining substrate):ketose (product):aldose (product) at the equilibrium point.

(7) Isomerase Reaction between D-Psicose and D-Allose

The D-psicose to D-allose isomerase reaction was then examined.

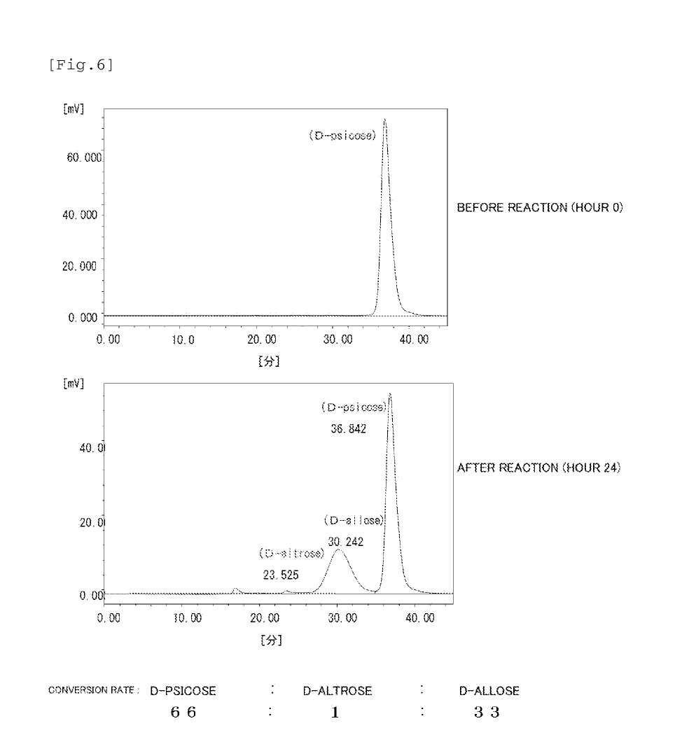

Enzyme reactions were performed under the reaction conditions of Table 9, and the sugar composition was measured by HPLC. FIG. 6 represents the result of the HPLC analysis before (hour 0, D-psicose) and after (a D-psicose, D-altrose, and D-allose mixture after 24-hour reaction) the reaction.

It was confirmed that the enzyme can generate D-allose from D-psicose in the D-psicose:D-allose:D-altrose ratio of 66:33:1 in the enzyme reaction performed by using D-psicose as a substrate. This was in conformity with the conversion rate given above for each aldose substrate.

TABLE 9
Reaction Conditions

| | | |
|---|---|---|
| Buffer (Gly-NaOH) pH: 9.0 | 200 | μL |
| Substrate (0.2M D-psicose) | 375 | μL |
| Enzyme solution (0.10 mg/50 μL) | 100 | μL |
| Coenzyme (0.01M MnCl₂) | 75 | μL |
| Temperature | 55° | C. |
| Reaction time | 24 | hours |

### CONCLUSION

The both enzymes had high affinity to L-rhamnose, though the activity was slightly higher in the XGP-720 enzyme than in the XGP-710 enzyme. These enzymes have an optimum temperature and stability in the vicinity of 60° C., which should be effective at preventing microorganism contamination. The enzymes showed the highest activity in a pH 9.0 glycine-sodium hydroxide buffer in the presence of MnCl₂ metal ions. These results clearly suggest that the enzymes of the Streptomyces bacterial strains identified herein are effective for the production of D-allose from D-psicose.

### INDUSTRIAL APPLICABILITY

The enzyme of the present invention is capable of producing D-allose through isomerization of D-psicose, and the microorganisms that produce this enzyme are strains of genus Streptomyces. Streptomyces microorganisms have been used in food production, and are considered safe. The most notable feature of using these enzyme-producing Streptomyces strains in food industry is the safety of the bacteria. Enzymes from microorganisms of genera Agrobacterium, Rhizobium, and Pseudomonas are known to produce D-allose and other rare sugars. However, these are reported as being opportunistic or causative of plant cell infection, and require a large labor force to check safety. The present invention enabling use of highly safe bacteria or bacterial culture is a large technological advancement. The present invention providing the enzyme capable of producing D-allose through isomerization of D-psicose, and the established method of production of such enzymes is thus considered to be industrially highly significant not only in sugar production but in related food, cosmetic, and drug industries.

1. A protein of any of the following (a) to (c) with the activity to recognize and react with the C1 CHO group and the C2 OH group of an aldose, and convert the C1 CHO group to an OH group and the C2 OH group to a CO group, or the activity to recognize and react with the C1OH group and the C2 CO group of a ketose, and convert the C1OH group to a CHO group and the C2 CO group to an OH group,
(a) a protein comprising the amino acid sequence represented by SEQ ID NO: 1 or SEQ ID NO: 2,
(b) a protein comprising the amino acid sequence represented by SEQ ID NO: 1 or SEQ ID NO: 2 with the substitution, addition, insertion, or deletion of one or several amino acid residues,
(c) a protein comprising an amino acid sequence that is at least 60% homologous to the amino acid sequence represented by SEQ ID NO: 1 or SEQ ID NO: 2,
wherein the protein is immobilized on a carrier.

2. The protein according to claim 1, wherein the protein isomerizes a ketose D-psicose to an aldose D-allose.

3. The protein according to claim 1, wherein the protein is specified by the following physical and chemical properties (d) to (f),
(d) an effective pH of 6.0 to 11.0, and an optimum pH of 9.0,
(e) an effective temperature of 10 to 80° C., and an optimum temperature of 60° C., and
(f) reactivity to L-rhamnose, D-xylose, D-ribose, D-allose, D-glucose, and L-arabinose.

4. A DNA fragment of Streptomyces sp. 710 (Deposition Number: NITE BP-01423) or Streptomyces sp. 720 (Deposition Number: NITE BP-01424) origin comprising the base sequence of SEQ ID NO: 3 or SEQ ID NO: 4, a complementary sequence thereof, or a sequence with a part of or all of the base sequence or the complementary sequence, and encoding a protein of any of the following (a) to (c) with the activity to recognize and react with the C1 CHO group and the C2 OH group of an aldose, and convert the C1 CHO group to an OH group and the C2 OH group to a CO group, or the activity to recognize and react with the C1 OH group and the C2 CO group of a ketose, and convert the C1OH group to a CHO group and the C2 CO group to an OH group,
(a) a protein comprising the amino acid sequence represented by SEQ ID NO: 1 or SEQ ID NO: 2,
(b) a protein comprising the amino acid sequence represented by SEQ ID NO: 1 or SEQ ID NO: 2 with the substitution, addition, insertion, or deletion of one or several amino acid residues,
(c) a protein comprising an amino acid sequence that is at least 60% homologous to the amino acid sequence represented by SEQ ID NO: 1 or SEQ ID NO: 2.

5. A recombinant vector comprising the DNA fragment of claim 4.

6. A host cell comprising an expression system capable of causing expression of the protein of claim 1.

7. A method for producing a recombinant protein,
the method comprising culturing the host cell with the expression system of claim 6 in a medium, and collecting the recombinant protein of claim 1 from the obtained culture.

8. A method for producing D-allose,
the method comprising isomerizing D-psicose to D-allose under the activity of the protein of claim 1.

9. The method for producing D-allose according to claim 8, wherein the D-psicose is produced by epimerizing D-fructose.

10. The method for producing D-allose according to claim 8, wherein the D-psicose is produced by directing D-glucose to D-fructose through isomerization, and epimerizing the D-fructose.

11. The method for producing D-allose according to claim 8, wherein the D-psicose is produced by obtaining D-glucose from an unused resource, directing the D-glucose into D-fructose through isomerization, and epimerizing the D-fructose.

12. The method for producing D-allose according to claim 8, wherein the target product D-allose is a mixture of D-psicose and D-allose.


SEQUENCE LISTING (8 sequences)

SEQ ID NO: 1 - protein, 386 aa, Streptomyces 710
MTELAAVKAALKTQAVETPSWAYGNSGTRFKVFAQPGVPRDPFEKLDDAAKVHEFTGAAPTVALHIPWDRVEDYAALAAHAEKRGVRIGAINSNTFQDDDYRLGSICHPDAAVRRKAVDHLLECVDIMDATGSRDLKLWFADGTNYPGQDDIRSRQDRLAEGLAEVYERLGEGQRMLLEYKLFEPAFYTTDVPDWGTAYAHCLKLGEKAQVVVDTGHHAPGTNIEFIVATLLREGKLGGFDFNSRFYADDDLMVGAADPFQLFRIMYEVVRGGGFTSDVAFMLDQCHNIEAKIPAIIRSVMNVQEATAKALLVDGTALAEAQAAGDVLEANAVLMDAYNTDVRPLLREVREESGLDPEPMKAYRSCGWAEKVVAERIGGQQAGWGA

SEQ ID NO: 2 - protein, 386 aa, Streptomyces 720
MTELAAVKAALKTQAVETPSWAYGNSGTRFKVFAQPGVPRTPQEKLDDAAQVHAYTGAAPTVALHIPWDRVEDYAKLAEYAEERGLKLGTINSNTFQDDAYKLGSICHPEAAVRRKALDHLLECVDIMDATGSKDLKLWFADGTNYPGQDDVRERQDRLTEGLATVYERLGDDQRMLLEYKFFEPAFYTTDVPDWGTAYAHCLKLGDRAQVVVDTGHHAPGTNIEFIVATLLREGKLGGFDFNSRFYADDDLMVGAADPFQLFRIMYEVIRGGGLTSDVAFMLDQCHNIEAKIPAIIRSVMNVQEATAKALLVDREALRSAQRSGDVLEANAVLMDAYNTDVRPLLAELREETGLDPDPVAAYRRSGWASKIAEERVGGQQAGWGA

SEQ ID NO: 3 - DNA, 1161 bp, Streptomyces 710
gtgaccgagctcgccgcggtgaaagccgcgctcaagacccaggccgtcgagacgccgtcgtgggcgtacggcaactcgggcacccgcttcaaggtgttcgcccagcccggtgtcccgcgcgaccccttcgagaaactggacgacgcggcgaaggtgcacgagttcaccggcgcggccccgaccgtcgccctgcacatcccgtgggaccgggtcgaggactacgccgcgctggcggcgcacgccgagaagcggggcgtgcggatcggcgcgatcaactccaacaccttccaggacgacgactacaggctgggcagcatctgccaccccgacgcggcggtgcgccggaaggccgtcgaccacctgctggagtgcgtcgacatcatggacgccaccggctcgcgcgacctgaagctgtggttcgccgacggcacgaactacccgggacaggacgacatccggtcccggcaggaccggctggccgagggcctggccgaggtgtacgagcggctcggcgaggggcagcggatgctcctggagtacaagctcttcgagccggcgttctacacgacggacgtgccggactggggcacggcgtacgcgcactgtctcaagctgggcgagaaggcccaggtcgtggtcgacacgggacaccacgcgccgggcacgaacatcgagttcatcgtggcgacgctgctgcgggaggggaagctcggcgggttcgacttcaactcgcggttctacgccgacgacgacctgatggtgggtgccgccgacccgttccagctgttccggatcatgtacgaggtggtgcgcgggggcgggttcacctccgacgtggcgttcatgctcgaccagtgccacaacatcgaggcgaagatcccggcgatcatccggtcggtgatgaacgtgcaggaggcgacggcgaaggcgctgctggtcgacggcaccgccctggccgaggcgcaggccgcgggggatgtgctggaggcgaacgcggtgctgatggacgcgtacaacacggacgtgcggccgctgctccgggaagtgcgggaggagtcggggctggaccccgaacccatgaaggcgtaccgctcctgcgggtgggcggagaaggtcgttgccgagcggatcggtgggcagcaggccgggtggggggcgtag

SEQ ID NO: 4 - DNA, 1161 bp, Streptomyces 720
gtgaccgagctcgccgcggtgaaggccgccctcaagacccaggccgtagagacgccgtcatgggcgtacgggaactcggggacccgcttcaaggtgttcgcgcagccgggagtcccccgcacgccccaggagaagctcgacgacgccgcgcaggtgcacgcgtacacgggtgccgcgccgaccgtggcactgcacatcccctgggaccgggtcgaggactacgcgaagctcgccgagtacgccgaggagcgcgggctgaagctggggacgatcaactccaacaccttccaggacgacgcgtacaagctcggcagcatctgccaccccgaggcggccgtgcgccgcaaagcccttgatcacttgctcgagtgcgtcgacatcatggacgcgaccggttcgaaggacctgaagctgtggttcgcggacggcaccaactatcccggccaggacgatgtccgtgagcgccaggaccgcctcaccgaagggctggccacggtgtacgagcggctcggtgacgaccagcggatgctgctcgagtacaagttcttcgagcccgccttctacacgacggacgtgccggactggggcacggcgtacgcgcactgcctcaagctcggtgacagggcgcaggtcgtggtcgacaccgggcatcacgcacccggcaccaacatcgagttcatcgtggccacactgctgcgcgaggggaagctcggcggcttcgacttcaactcccgcttctacgccgacgacgacctgatggtgggcgccgccgaccccttccagctgttccggatcatgtacgaggtgatccgcggcggcggcctcacgtccgacgtggccttcatgctcgaccagtgccacaacatcgaggcgaagatcccggcgatcatccgctcggtgatgaacgtccaggaggcgacggcgaaggcgctgctcgtggatcgtgaggcgctgcggtcggcgcagcggtccggggacgtgctggaggcgaacgcggtcctcatggacgcgtacaacaccgatgtacggccgctgctcgccgagctgcgcgaggagacggggctcgacccggacccggtcgccgcgtaccggcggtccgggtgggcgtcgaagatcgccgaggagcgggtgggtggtcagcaggcgggctggggagcgtga

SEQ ID NO: 5 - DNA, 30 bp, Artificial Sequence
  primer
ggaattccatatgaccgagctcgccgcggt

SEQ ID NO: 6 - DNA, 30 bp, Artificial Sequence
  primer
cccaagcttctacgccccccacccggcctg

SEQ ID NO: 7 - DNA, 30 bp, Artificial Sequence; identical to SEQ ID NO: 5
  primer

SEQ ID NO: 8 - DNA, 30 bp, Artificial Sequence
  primer
cccaagctttcacgctccccagcccgcctg